(19) United States
(12) Patent Application Publication
(10) Pub. No.: US 2020/0289710 A1
(43) Pub. Date: Sep. 17, 2020

(54) PRODUCTS OF MANUFACTURE HAVING ENHANCED BIOCOMPATIBILITY AND ANTIBACTERIAL PROPERTIES AND METHODS OF MAKING AND USING THEM

(71) Applicant: UNIVERSITY OF NORTH TEXAS, Denton, TX (US)

(72) Inventors: Donghui ZHU, Frisco, TX (US); Yingchao SU, Denton, TX (US)

(21) Appl. No.: 16/815,891

(22) Filed: Mar. 11, 2020

Related U.S. Application Data
(60) Provisional application No. 62/816,638, filed on Mar. 11, 2019.

(51) Int. Cl. A61L 27/46; A61L 27/04; A61L 27/06; A61L 27/44

(52) U.S. Cl. CPC A61L 27/46; A61L 27/042; A61L 27/06; A61L 2400/18; A61L 27/446; A61L 2420/04; A61L 27/047

(57) ABSTRACT

In alternative embodiments, provided are products of manufacture such as medical or dental devices, e.g., bone implants, having zinc phosphate (ZnP) coatings prepared on zinc (Zn), magnesium (Mg), and iron (Fe) based biodegradable metals and other non-biodegradable substrates, e.g., stainless steel, titanium and its alloys, cobalt-chrome alloys, nickel titanium alloys, to improve surface biocompatibility and provide antibacterial properties, and to enhance vascularization, and methods of making and using them. In alternative embodiments, also provided are methods to form ZnP coatings, including ZnP coatings with a porous surface, on metal surfaces such as zinc surfaces, and Zn-, Mg-, and Fe-based biodegradable metals, and other non-biodegradable substrates.

### RELATED APPLICATIONS

This U.S. Utility patent application claims the benefit of priority under 35 U.S.C. § 119(e) of U.S. Provisional Application Ser. No. 62/816,638 filed Mar. 11, 2019. The aforementioned application is expressly incorporated herein by reference in its entirety and for all purposes.

### STATEMENT AS TO FEDERALLY SPONSORED RESEARCH

This invention was made with government support under National Institutes of Health (NIH), DHHS, grant no. R01HL140562. The government has certain rights in the invention.

### TECHNICAL FIELD

This invention generally relates to medical or dental devices such as bone implants. In alternative embodiments, provided are products of manufacture such as medical or dental devices, e.g., bone implants, having zinc phosphate (ZnP) coatings prepared on zinc (Zn), magnesium (Mg), and iron (Fe) based biodegradable metals and other non-biodegradable substrates, e.g., stainless steel, titanium and its alloys, cobalt-chrome alloys, nickel titanium alloys, to improve surface biocompatibility and provide antibacterial properties, and to enhance vascularization, and methods of making and using them. In alternative embodiments, also provided are methods to form ZnP coatings, including ZnP coatings with a porous surface, on metal surfaces such as zinc surfaces, and Zn-, Mg-, and Fe-based biodegradable metals, and other non-biodegradable substrates.

### BACKGROUND

Metallic implants play significant roles in the clinical treatment and therapy for the coronary artery and orthopedic surgery¹⁻³. Traditional metallic implants have been applied as coronary stents, orthopedic scaffolds, and bone plates and screws, etc. They have numerous advantages, including good machinability for complex structures, low risks of restenosis, and high mechanical support and durability^{1, 4}. However, there are non-ignorable serious side effects faced by the traditional metallic implants. Long term anti-clotting medicine is required to reduce the thrombosis risks for the inert stents, while a second removal surgery is necessary when the orthopedic tissue has recovered^{5, 6}. In addition, chronic inflammation is a common concern for the long-term permanent implants^{1, 6, 7}.

Compared to the conventional metallic implant materials, biodegradable metals as temporary implants have been developed to avoid a secondary surgery, thereby accelerating the entire healing process while simultaneously reducing health risks, costs and scarring^{1, 5}. Up to now, magnesium (Mg), iron (Fe) and zinc (Zn) are the three main classes of biodegradable metals as functional but temporary implants^{1, 5, 8-11}. Zn is considered a promising biodegradable metal thanks to its essential role in many enzymes and in cell metabolic activity and functions¹²⁻¹⁴. In addition, it has a probably more suitable degradation rate, which is more likely in line with the clinical demand^{15, 16}.

However, one of the significant concerns about Zn as a degradable metal is its local and systemic toxicity; the recommended dietary allowance (RDA) for Zn is only 15-40 mg/day, much lower than that of Mg (300-400 mg/day)¹⁷. Moreover, notable cytotoxicity of Zn has been reported in different cells, including human bone cells¹⁸⁻²⁰ and vascular cells^{20, 21}.

To improve the surface biocompatibility of implants, calcium phosphate (CaP) has been used. CaP owns the inherent bone tissue compatibility due to their similar composition to carbonated apatite in natural bone tissue²². Therefore, CaP is applied in the orthopedic applications in the form of ceramic substrates, reinforcement in composites, bone cement, or surface bio-functional coating²³⁻³⁰.

As a natural phosphate of Zn-based metals, ZnP has stable chemical properties and shows biocompatibility³¹⁻³². The feasibility of ZnP coating has been explored on several biomedical metallic substrates, including Ti, Fe and Mg alloys, but not Zn-based ones³³⁻³⁵. ZnP coating has been shown to modify the degradation rate of biodegradable Fe- and Mg-based alloys³⁴⁻³⁵ and promote a fibroblast cell's adhesion on a Ti alloy³³. Zn ion released from a Zn-based material could potentially interact with a bacteria surface to induce cell deformation and bacteriolysis³⁶.

### SUMMARY

In alternative embodiments, provided are products of manufacture comprising a zinc phosphate (ZnP) or a ZnP-based composite coating, or a combination of a ZnP coating and a ZnP-based composite coating, on or deposited on:
(a) a zinc (Zn) metal surface, or a Zn-based metal or Zn alloy surface;
(b) a magnesium (Mg) or an iron (Fe) surface, or an Mg-based or an iron (Fe)-based metal surface or Mg or Fe alloy surface; or,
(c) a nondegradable metal or nondegradable metal alloy surface, wherein optionally the nondegradable metal or nondegradable metal alloy surface comprises stainless steel, a titanium (Ti) or Ti alloy, a cobalt-chrome alloy, a nickel titanium alloy, or a combination thereof.

In alternative embodiments, for products of manufacture as provided herein:
the products of manufacture are manufactured as a medical or a dental device, wherein optionally the device is a bone implant or a prosthetic, or the device is an orthopedic, dental, craniofacial or cardiovascular device, or is used for a biomedical application, wherein optionally the biomedical application is an orthopedic, dental, craniofacial or cardiovascular application;
the ZnP or the ZnP-based composite coating is porous or non-porous, or a combination thereof, wherein optionally the porous ZnP surface is made or deposited by a process comprising hydrothermal, anodization, electrochemical deposition or any combination thereof; and optionally the non-porous ZnP coating is made or deposited by a process comprising spray deposition, pulsed laser deposition, sputter deposition or any combination thereof;
the ZnP coating or the ZnP-based composite coating is (or averages): between about 0.5 μm to 100 μm, or between about 1.0 μm to 50 μm, where the average ZnP or ZnP-based composite coating thickness on a surface using a chemical method of deposition is between about 3 μm to 6 μm;
the ZnP coating or the ZnP-based composite coating covers between about 1% to 100%, or between about 10% and 90%, or between about 20% and 80%, of the surface of the product of manufacture;
the ZnP-based composite coating comprises one or more other (non-Zn) inorganic coating compositions, wherein optionally the one or more other (non-Zn) inorganic coating compositions comprise zinc oxide, zinc hydroxide, a calcium phosphate, a hydroxyapatite (HA), or an equivalent or a mixture thereof,

wherein optionally the inorganic component of the ZnP composite coating comprises between about 1% to 95%, or between about 10% and 90%, or between about 20% and 80%, by weight, mass or molar ratio of the ZnP composite coating (the remainder being ZnP);
the ZnP-based composite coating comprises one or more organic compounds, herein optionally the one or more organic compounds comprise a collagen, chitosan, gelatin, hyaluronic acid, polylactic acid, polylactide, or an equivalent or a mixture thereof,

wherein optionally the organic component of the ZnP composite coating comprises between about 1% to 95%, or between about 10% and 90%, or between about 20% and 80%, by weight, mass or molar ratio of the ZnP composite coating (the remainder being ZnP);
the ZnP coating or the ZnP-based composite coating is a multi-layered structure, wherein optionally the inner layer of the coating comprises a Mg alloy, wherein optionally the Mg alloy comprises magnesium hydroxide and/or magnesium phosphate;
the products of manufacture further comprise a cell, wherein optionally the cell is a pre-osteoblast or an osteoblast;
the Zn alloy comprises aluminum, iron, magnesium, calcium, strontium, silver, copper, titanium, manganese or lithium or any combination thereof; and optionally the Zn alloy has a proportion of the following compositions:
   from zero to about 12.0 weight percent of aluminum, or between about 0 to about 25 weight percent of aluminum,
   from zero to about 10.0 weight percent of magnesium, or between about 0 to about 20 weight percent of magnesium,
   from zero to about 10.0 weight percent of calcium, or between about 0 to about 20 weight percent of calcium,
   from zero to about 10.0 weight percent of strontium, or between about 0 to about 20 weight percent of strontium,
   from zero to about 8.0 weight percent of silver, or between about 0 to about 20 weight percent of silver,
   from zero to about 8.0 weight percent of copper, or between about 0 to about 20 weight percent of copper,
   from zero to about 5.0 weight percent of titanium, or between about 0 to about 20 weight percent of titanium,
   from zero to about 5.0 weight percent of manganese, or between about 0 to about 20 weight percent of manganese,
   from zero to about 5.0 weight percent of lithium, or between about 0 to about 20 weight percent of lithium,
and a balance of zinc, based on the total weight of the composition;
   the magnesium (Mg) alloy comprises aluminum, zinc, calcium, strontium, silver, copper, titanium, manganese or lithium or any combination thereof; and optionally has a proportion of the following compositions:
   from zero to about 12.0, or between about 0 to about 25, weight percent of aluminum,
   from zero to about 10.0, or between about 0 to about 20, weight percent of zinc,
   from zero to about 10.0, or between about 0 to about 20, weight percent of calcium,
   from zero to about 10.0, or between about 0 to about 20, weight percent of strontium,
   from zero to about 8.0, or between about 0 to about 20, weight percent of silver,
   from zero to about 8.0, or between about 0 to about 20, weight percent of copper,
   from zero to about 5.0, or between about 0 to about 20, weight percent of titanium,
   from zero to about 5.0, or between about 0 to about 20, weight percent of manganese,
   from zero to about 5.0, or between about 0 to about 20, weight percent of lithium,
and a balance of zinc, based on the total weight of the composition;
   the iron (Fe) alloy comprises aluminum, zinc, calcium, strontium, silver, copper, titanium, manganese or lithium or any combination thereof; and optionally has a proportion of the following compositions:
   from zero to about 40.0, or between about 0 to about 60, weight percent of manganese,
   from zero to about 10.0, or between about 0 to about 20, weight percent of cobalt,
   from zero to about 8.0, or between about 0 to about 20, weight percent of aluminum,
   from zero to about 8.0, or between about 0 to about 20, weight percent of tungsten,
   from zero to about 8.0, or between about 0 to about 20, weight percent of tin,
   from zero to about 8.0, or between about 0 to about 20, weight percent of boron,
   from zero to about 8.0, or between about 0 to about 20, weight percent of carbon,
   from zero to about 5.0, or between about 0 to about 20, weight percent of sulfur,
   from zero to about 5.0, or between about 0 to about 20, weight percent of silicon,
and a balance of zinc, based on the total weight of the composition; and/or
   the ZnP content of the ZnP-comprising coatings is between about 50.1% to 100%, or between about 55% and 95%, or between about 60% and 90%, by weight of the ZnP-comprising coating.

In alternative embodiments, provided are kits comprising a product of manufacture as provided herein.

In alternative embodiments, provided are use of: a product of manufacture as provided herein, or a kit as provided herein, for: increasing cell adhesion to the product of manufacture in situ or in vivo, enhancing vascularization in situ or in vivo, or increasing the rate of osteogenic differentiation of pre-osteoblasts to osteoblasts.

In alternative embodiments, products of manufacture and/or kits as provided herein are used for: increasing cell adhesion to the product of manufacture in situ or in vivo, enhancing vascularization in situ or in vivo, or increasing the rate of osteogenic differentiation of pre-osteoblasts to osteoblasts.

In alternative embodiments, provided are methods for: increasing cell adhesion to a product of manufacture in situ or in vivo, enhancing vascularization in situ or in vivo, or increasing the rate of osteogenic differentiation of pre-osteoblasts to osteoblasts, comprising: implanting in vivo a product of manufacture as provided herein.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A-F illustrate images of surface ZnP coating morphology and phase composition: coatings formed at: pH=2, the inset bar indicating a length of 50 μm (FIG. 1A); pH=2, the inset bar indicating a length of 10 μm (FIG. 1B); pH=2.5, the inset bar indicating a length of 50 μm (FIG. 1C); pH=2.5, the inset bar indicating a length of 10 μm (FIG. 1D); and, pH=3, the inset bar indicating a length of 50 μm (FIG. 1E), pH=3, the inset bar indicating a length of 10 μm (FIG. 1E);

FIG. 1G graphically illustrates elemental compositions (EDS), where the atomic composition percentage (%) is a function of pH, with a Zn control, where Zn is black, oxygen (O) is red, and phosphorus (P) is blue; and,

FIG. 1H graphically illustrates X-ray diffraction pattern (XRD) patterns, where intensity is a function of 2-theta,

as discussed in detail in Example 1, below.

FIG. 2A-C illustrate electrochemical corrosion behaviors of an uncoated and ZnP-coated Zn metal substrate: FIG. 2A graphically illustrates potentio-dynamic polarization, where current density is a function of potential; FIG. 2B graphically illustrates electrochemical impedance spectroscopy; and, FIG. 2C illustrates a table of electrochemical corrosion parameters, as discussed in detail in Example 1, below.

FIG. 3A-F illustrate degraded surface morphology and compositions of ZnP-coated surfaces and non-coated surfaces after two months of immersion test in the Hanks' solution: FIG. 3A and FIG. 3C illustrates a general view at 1 mm and 10 um, respectively; and, FIG. 3B and FIG. 3D illustrate a magnified view of FIG. 3A and FIG. 3B, showing uncoated Zn surface; and FIG. 3C and FIG. 3D, showing an exemplary ZnP coated surface; and, the corresponding X-ray diffraction pattern (XRD) patterns FIG. 3E; and, FIG. 3F, graphically illustrating pH evolution with immersion time comparing Zn and ZnP-coated surfaces, as discussed in detail in Example 1, below.

FIG. 4A-F illustrate the hemocompatibility of ZnP-coated surfaces and non-coated surfaces samples: FIG. 4A-D illustrate images of platelets adhesion morphology on FIG. 4A, the inset bar indicating a length of 20 μm, and FIG. 4B, the inset bar indicating a length of 5 μm, for pure Zn; and FIG. 4C (the inset bar indicating a length of 20 μm) and FIG. 4D (the inset bar indicating a length of 5 μm) for a ZnP coating, and FIG. 4E graphically illustrating the corresponding number of adhered platelets; and FIG. 4F graphically illustrating hemolysis percentage, as discussed in detail in Example 1, below.

FIG. 5A-B graphically illustrate data from an MTT (3-(4,5-dimethylthiazol-2-yl)-2,5-diphenyltetrazolium bromide) assay for cell viability: (FIG. 5A) pre-osteoblasts and (FIG. 5B) endothelial cells cultured with extract media prepared by incubation with samples for 72 hours (h), as discussed in detail in Example 1, below.

FIG. 6A-H illustrate images showing cell adhesion morphology of (FIG. 6A (the inset bar indicating a length of 50 μm), FIG. 6B (the inset bar indicating a length of 5 μm), FIG. 6E (the inset bar indicating a length of 50 μm), FIG. 6F (the inset bar indicating a length of 5 μm) pre-osteoblasts and (FIG. 6C (the inset bar indicating a length of 50 μm), FIG. 6D (the inset bar indicating a length of 10 μm), FIG. 6G (the inset bar indicating a length of 50 μm), FIG. 6H (the inset bar indicating a length of 10 μm) endothelial cells with different samples for 72 h: (FIG. 6A-D) illustrate images of uncoated Zn, FIG. 6E-H illustrate images of ZnP coatings, as discussed in detail in Example 1, below.

FIG. 7A-G illustrate images of different cell differentiation behaviors of pre-osteoblasts cultured with different sample extracts, depending on whether the substrate was coated or uncoated: FIG. 7A-C illustrate alkaline phosphatase (ALP) staining; FIG. 7D graphically illustrates ALP activity; FIG. 7E-G illustrate alkaline red staining: where FIG. 7A and FIG. 7E are negative controls, FIG. 7B and FIG. 7F show uncoated Zn, and FIG. 7C and FIG. 7G show ZnP coating, as discussed in detail in Example 1, below.

FIG. 8A-C illustrate antibacterial performance of different samples cultured with E. coli for 24 hours (h); bacterial adhesion on surfaces of: FIG. 8A illustrates uncoated Zn coating, FIG. 8B illustrates a ZnP coating, and FIG. 8C graphically illustrates the corresponding number of adhered bacterial cells, as discussed in detail in Example 1, below.

Like reference symbols in the various drawings indicate like elements.

### DETAILED DESCRIPTION

In alternative embodiments, provided are products of manufacture such as medical or dental devices, e.g., implants such as bone implants, having zinc phosphate (ZnP) coatings prepared on zinc- (Zn-), magnesium- (Mg-), and iron- (Fe)-based biodegradable metals and other non-biodegradable substrates, e.g., stainless steel, titanium and its alloys, cobalt-chrome alloys, nickel titanium alloys, to improve surface biocompatibility of the medical or dental devices, e.g., implants, with cells such as pre-osteoblasts; and, to provide antibacterial properties, and methods of making and using them. In alternative embodiments, also provided are methods for making the ZnP coatings, e.g., on metal (e.g., zinc) surfaces, such as zinc (Zn)-based biodegradable or biocompatible metals and other substrates. In alternative embodiments, provided are products of manufacture such as medical or dental devices, e.g., implants comprising a ZnP coating on a metal surface, e.g., a Zn metal surface, or other biodegradable metal surfaces, to improve the biocompatibility and antibacterial properties. In alternative embodiments, provided are products of manufacture as provided herein are used for orthopedic and/or vascular applications.

In alternative embodiments, methods as provided herein comprise optimization or modification of the ZnP coating morphology by controlling or modifying the pH and compositions of coating solutions for different substrates; the result being that methods as provided herein can produce a homogeneous micro-/nano-ZnP coating structure or surface, e.g., on a metal surface, or on a product of manufacture surface, e.g., on an implant surface. In alternative embodiments, ZnP coatings as provided herein or ZnP coatings made by methods as provided herein result in: significantly increased the cell (e.g., pre-osteoblast or osteoblast) viability; increased cell adhesion to a product of manufacture surface, e.g., an implant surface; and/or, increased or faster rate of osteogenic differentiation of pre-osteoblasts to osteoblasts. In alternative embodiments, ZnP coatings as provided herein or ZnP coatings made by methods as provided herein result in: enhanced vascular cell attachment to a product of manufacture surface, e.g., an implant surface; and/or, increased cell growth and proliferation. In alternative embodiments, ZnP coatings as provided herein or ZnP coatings made by methods as provided herein result in: a reduction of platelet adhesion to a product of manufacture surface, e.g., an implant surface, and a reduction in platelet activation. In alternative embodiments, ZnP coatings as provided herein or ZnP coatings made by methods as provided herein have anti-bacterial properties, e.g., to reduce potential infection after an implantation of a medical or dental device.

In alternative embodiments, provided are products of manufacture such as medical or dental devices, e.g., implants such as bone implants, having a ZnP coating on a biodegradable metal alloy, e.g., a Zn based biodegradable metal alloys or other biodegradable metal alloy, including but not limited to Mg-based biodegradable metal alloys and Fe-based biodegradable metal alloys.

In alternative embodiments, provided are products of manufacture such as medical or dental devices, e.g., implants such as bone implants, having a ZnP coating on a biocompatible alloy, e.g., an inert biomedical metallic alloy such as a stainless steel, titanium and its alloys, cobalt-chrome alloys and/or nickel titanium alloys.

In alternative embodiments, provided are products of manufacture having a coating that is partly, substantially or completely comprising a ZnP coating; for example, a product of manufacture as provided herein can cover between about 1% to 100%, or between about 10% and 90%, of the surface of the product of manufacture, e.g., biomedical or dental device such as an implant. For example, products of manufacture as provided herein can be a zinc phosphate-based composite (i.e., a mixed ingredient) coating comprising one or more other inorganic coating compositions, e.g., comprising zinc oxide, zinc hydroxide, calcium phosphates and/or hydroxyapatite (HA), and the like, or mixtures thereof. In alternative embodiments, provided are products of manufacture having a ZnP based coating that is a composite with an organic compound, e.g., an organic coating composition such as a collagen, chitosan, gelatin, hyaluronic acid, polylactic acid and/or polylactide, and the like, or mixtures thereof. In alternative embodiments, the inorganic and/or organic component of the coating can comprise between about 1% to 95%, or between about 10% and 90%, or between about 20% and 80%, by weight, mass or molar ratio of the coating (the remainder being ZnP).

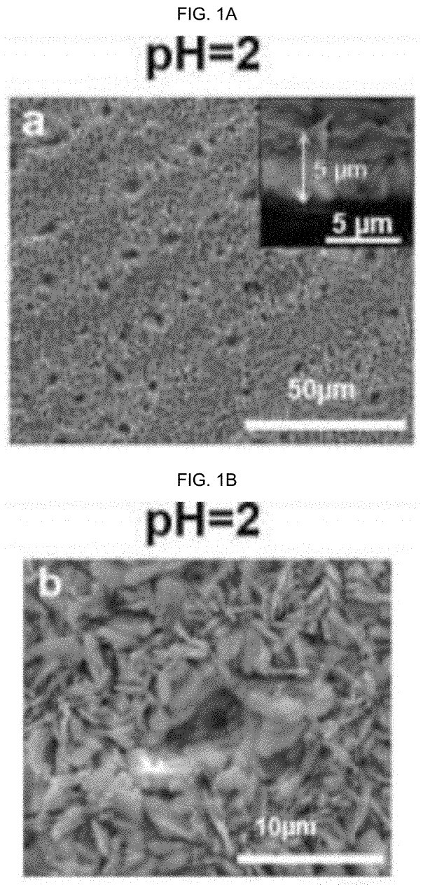

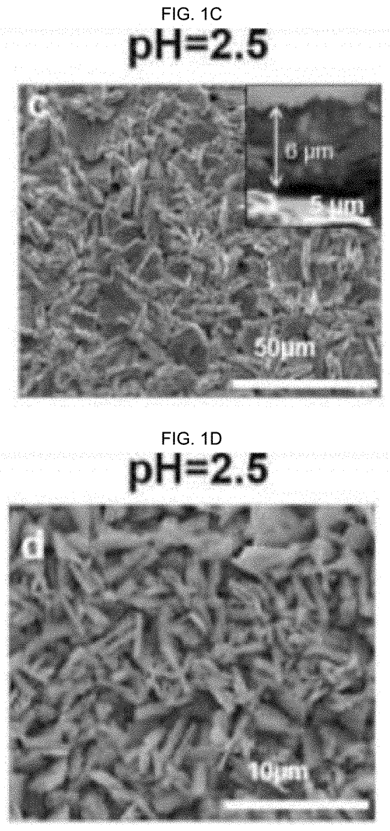

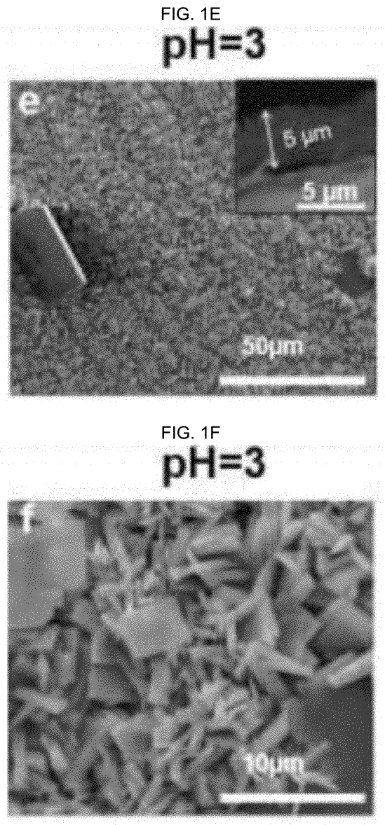

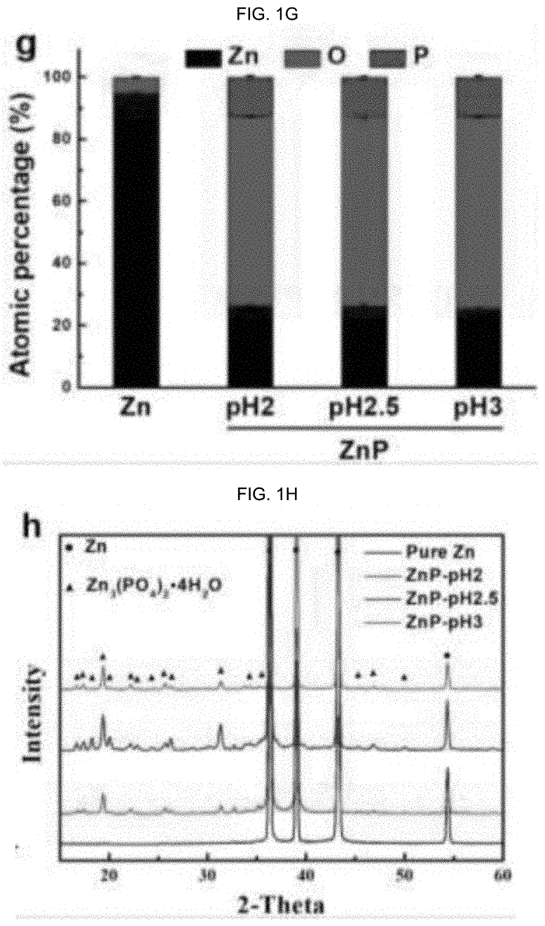

In alternative embodiments, a Zn coating is between about 0.5 to 100 μm, or between about 1.0 to 50 μm, where the average Zn coating thickness on a Zn metal using a chemical method of deposition is between about 3 to 6 μm, as shown in FIG. 1. The thickness of the Zn coating can differs when using different coating methods and different substrates. The coating time is the most effective way to control the thickness, and the coating solution concentration, temperature, pH values are also modified to control thickness.

In alternative embodiments, Zn coatings are multi-layered, e.g., Zn coatings can comprise other coatings to form a composite coating. In alternative embodiments, a metallic substrate can affect a coating composition in a multi-layered structure, e.g. the inner layer of a coating can comprise a Mg alloy, where the Mg alloy can comprise magnesium hydroxide and/or magnesium phosphate.

In alternative embodiments, the ZnP based coatings as provided herein can significantly improve the biocompatibility of the product of manufacture. For example, the ZnP based coatings as provided herein are cyto-compatible and can provide improved or enhanced cell (e.g., pre-osteoblast or osteoblast) adhesion to the product of manufacture; and use of ZnP based coatings as provided herein can result in improved or enhanced cell viability, proliferation and differentiation. In alternative embodiments, the ZnP based coatings as provided herein are hemo-compatible, resulting in decreased platelet adhesion and hemolysis rate.

In alternative embodiments, the ZnP based coatings as provided are antibacterial, and can provide or significantly improve on antibacterial properties, for example, by decreasing or preventing the surface adhesion of the bacteria, thereby decreasing the surrounding bacterial numbers. In alternative embodiments, the ZnP based coatings are antibacterial against Staphylococcus, e.g., Staphylococcus aureus, and/or gram negative bacteria, e.g., Escherichia coli.

In alternative embodiments, the ZnP based coatings as provided act as a biomedical coating on a biodegradable or a non-biodegradable metal or metal alloy.

In alternative embodiments, ZnP-comprising surface coatings have enhanced biocompatibility in vivo, e.g., with cells, e.g., they enhance vascularization, and promote cell adhesion and osteoblast differentiation.

In alternative embodiments, ZnP-comprising surface coatings as provided herein have antibacterial properties.

In alternative embodiments, ZnP-comprising surface coatings as provided herein and products of manufacture as provided herein can be used in biomedical applications such as in orthopedic, dental, craniofacial and cardiovascular applications and related surgeries.

In alternative embodiments, ZnP-comprising surface coatings are applied onto biodegradable metal alloys, which can comprise Zn-, Mg- and Fe-based biodegradable metal alloys.

In alternative embodiments, the Zn alloy can comprise aluminum, iron, magnesium, calcium, strontium, silver, copper, titanium, manganese or lithium or any combination thereof; and can have a proportion of the following compositions including but not limited to:
from zero to about 12.0 weight percent of aluminum, or between about 0 to about 25 weight percent of aluminum,
from zero to about 10.0 weight percent of magnesium, or between about 0 to about 20 weight percent of magnesium,
from zero to about 10.0 weight percent of calcium, or between about 0 to about 20 weight percent of calcium,
from zero to about 10.0 weight percent of strontium, or between about 0 to about 20 weight percent of strontium,
from zero to about 8.0 weight percent of silver, or between about 0 to about 20 weight percent of silver,
from zero to about 8.0 weight percent of copper, or between about 0 to about 20 weight percent of copper,
from zero to about 5.0 weight percent of titanium, or between about 0 to about 20 weight percent of titanium,
from zero to about 5.0 weight percent of manganese, or between about 0 to about 20 weight percent of manganese,
from zero to about 5.0 weight percent of lithium, or between about 0 to about 20 weight percent of lithium,

and a balance of zinc, based on the total weight of the composition.

In alternative embodiments, the magnesium (Mg) alloy can comprise aluminum, zinc, calcium, strontium, silver, copper, titanium, manganese or lithium or any combination thereof; and can have a proportion of the following compositions including but not limited to:
from zero to about 12.0, or between about 0 to about 25, weight percent of aluminum,
from zero to about 10.0, or between about 0 to about 20, weight percent of zinc,
from zero to about 10.0, or between about 0 to about 20, weight percent of calcium,
from zero to about 10.0, or between about 0 to about 20, weight percent of strontium,
from zero to about 8.0, or between about 0 to about 20, weight percent of silver,
from zero to about 8.0, or between about 0 to about 20, weight percent of copper,
from zero to about 5.0, or between about 0 to about 20, weight percent of titanium,
from zero to about 5.0, or between about 0 to about 20, weight percent of manganese,
from zero to about 5.0, or between about 0 to about 20, weight percent of lithium,

and a balance of zinc, based on the total weight of the composition.

In alternative embodiments, the iron (Fe) alloy can comprise aluminum, zinc, calcium, strontium, silver, copper, titanium, manganese or lithium or any combination thereof; and can have a proportion of the following compositions including but not limited to:
from zero to about 40.0, or between about 0 to about 60, weight percent of manganese,
from zero to about 10.0, or between about 0 to about 20, weight percent of cobalt,
from zero to about 8.0, or between about 0 to about 20, weight percent of aluminum,
from zero to about 8.0, or between about 0 to about 20, weight percent of tungsten,
from zero to about 8.0, or between about 0 to about 20, weight percent of tin,
from zero to about 8.0, or between about 0 to about 20, weight percent of boron,
from zero to about 8.0, or between about 0 to about 20, weight percent of carbon,
from zero to about 5.0, or between about 0 to about 20, weight percent of sulfur,
from zero to about 5.0, or between about 0 to about 20, weight percent of silicon,

and a balance of zinc, based on the total weight of the composition.

In alternative embodiments, ZnP-comprising coatings as provided herein, or the ZnP-comprising coatings used on the surface of products of manufacture as provided herein, have as a main or primary composition ZnP or a composite composition of ZnP with other inorganic and organic materials, including but not limited to: zinc oxide, zinc hydroxide, calcium phosphates, and hydroxyapatite (HA), and collagen, chitosan, gelatin, hyaluronic acid, polylactic acid, polylactide, and the like or mixtures thereof. In alternative embodiments, the ZnP content of the ZnP-comprising coatings as provided herein, or the ZnP-comprising coatings used on the surface of products of manufacture as provided herein, is between about 50.1% to 100%, or between about 55% and 95%, or between about 60% and 90%, by weight of the ZnP-comprising coating or the ZnP-comprising coating used on the surface of the product of manufacture.

In alternative embodiments, ZnP-comprising coatings as provided herein, or the ZnP-comprising coatings used on the surface of products of manufacture as provided herein, have a nonporous structure or a porous structure, depending on the different coating methods (a porous structure is formed automatically using certain coating method and is related to ceramic crystal nucleation and growth). A nonporous structure can improve the coating stability and improve the cell growth and compatibility. A micro- and/or nano-sized porous structure (which can be a homogeneous micro-/nano-sized porous structure) can improve the coating's adhesion properties to other inorganic and organic coating layers, biological substances or cells such as vascular cells, fibroblasts or osteoblasts.

In alternative embodiments, ZnP-comprising coatings as provided herein, or the ZnP-comprising coatings used on the surface of products of manufacture as provided herein, have a porous structure, e.g., a homogeneous micro- and/or nano-sized porous structure, to improve the ZnP-comprising coating's biocompatibility, for example: cytocompatibility to improve the cell adhesion, viability, proliferation, and differentiation, hemocompatibility to decrease the platelets adhesion and hemolysis rate, and the like.

ZnP-comprising coatings as provided herein, or porous ZnP-comprising coatings as provided herein, can be prepared and/or deposited on substrates using various methods and processes. For example, to produce porous coatings non-limiting examples of such processes include: hydrothermal, anodization, electrochemical deposition, and combinations thereof. To produce nonporous coatings, non-limiting examples of such processes include: spray deposition, pulsed laser deposition, sputter deposition, and combinations thereof to produce nonporous coatings.

### Kits

Provided are kits comprising products of manufacture as described herein, optionally including instructions for use comprising methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

### Example 1: Making and Using Exemplary Products of Manufacture

This example provides data demonstrating that the ZnP based coatings as provided herein are cyto-compatible and can provide improved or enhanced cell (e.g., pre-osteoblast or osteoblast) adhesion to the product of manufacture; and use of ZnP based coatings as provided herein can result in improved or enhanced cell viability, proliferation and differentiation. In alternative embodiments, the ZnP based coatings as provided herein are hemo-compatible, resulting in decreased platelet adhesion and hemolysis rate.

Non-porous or porous ZnP coatings as provided herein can be prepared and/or deposited on substrates using various methods and processes. For example, non-limiting examples of such processes include: hydrothermal, anodization, electrochemical deposition, and combinations thereof to produce porous coatings. Non-limiting examples of such processes include: spray deposition, pulsed laser deposition, sputter deposition, and combinations thereof to produce nonporous coatings.

One exemplary coating method comprises: Zn samples were polished using #1500 sandpaper, cleaned by sonication in acetone for 5 to 20 min, then immersed in coating solution (20-100 mL/cm² ratio to the sample surface area) at room temperature (RT) for 2 to 20 min followed by rinsing with deionized water and drying in air before characterization. The coating solution was composed of 0 to 0.1 M Zn(NO₃)₂ and 0.1 to 0.3 M H₃PO₄ and the pH value was adjusted to pH 2 to 3, respectively. The main coating characterizations are shown in FIG. 1.

FIG. 1A-H illustrates images of surface ZnP coating morphology and phase composition: coatings formed at (a, b) pH=2, (c, d) pH=2.5 and (e, f) pH=3, (g) elemental compositions (EDS), and (h) X-ray diffraction pattern (XRD) patterns.

In alternative embodiments, products of manufacture or coatings as provided herein are sterilized using, e.g., irradiation, e.g., gamma or beta irradiation, or by using chemicals, e.g., using alcohol, e.g., 75% alcohol solutions, or gases, e.g., ethylene oxide.

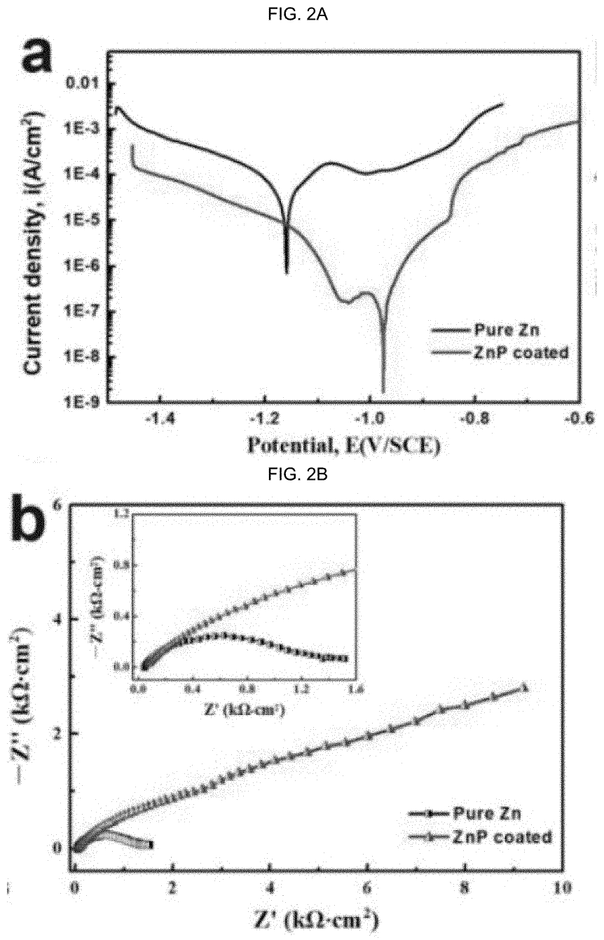

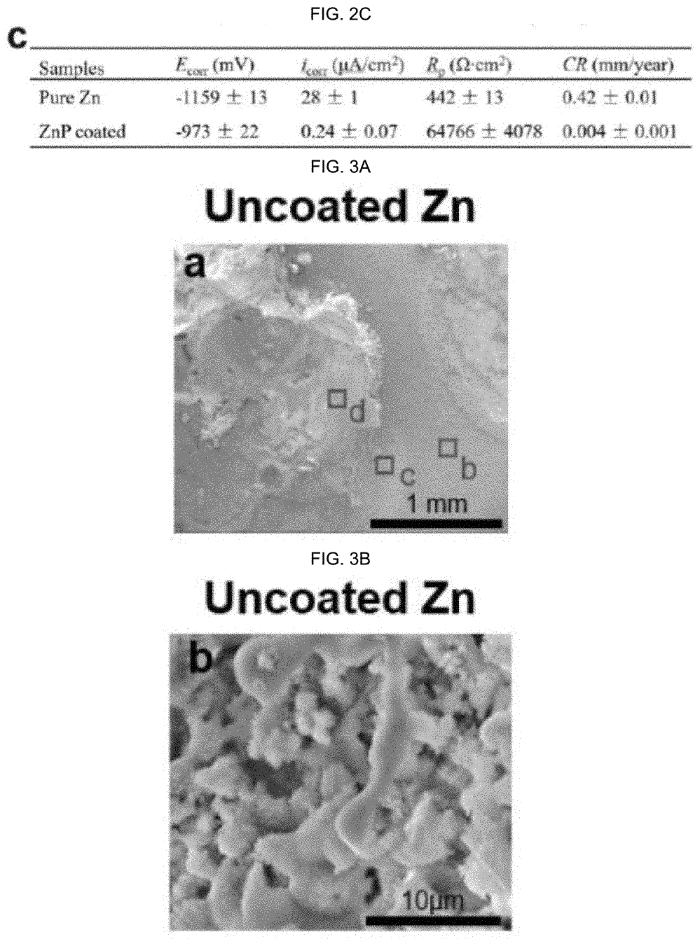

In alternative embodiments, products of manufacture or coatings as provided herein are corrosion resistant or have substantial corrosion resistance, e.g., to control the degradation rate of the biodegradable metal alloys and protect the inert metal alloys from degradation. A coating as provided herein can decrease the corrosion rate of an product of manufacture of an implant placed in vivo by between about 1 to 3 orders of magnitude, see e.g., FIG. 2. FIG. 2A-C illustrate electrochemical corrosion behaviors of an uncoated and ZnP-coated Zn metal substrate: (a) graphically illustrates potentio-dynamic polarization, (b) graphically illustrates electrochemical impedance spectroscopy, (c) illustrates a table of electrochemical corrosion parameters.

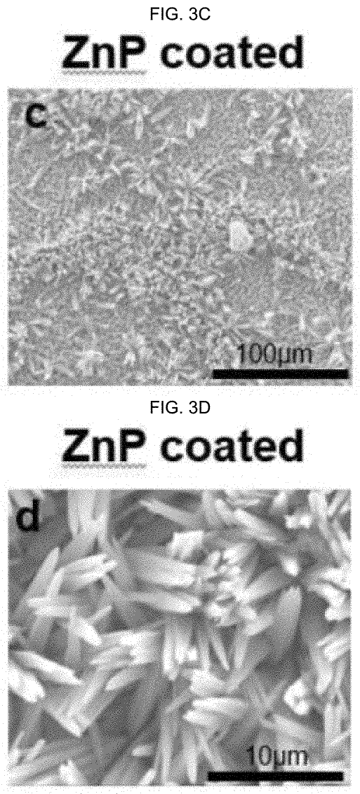

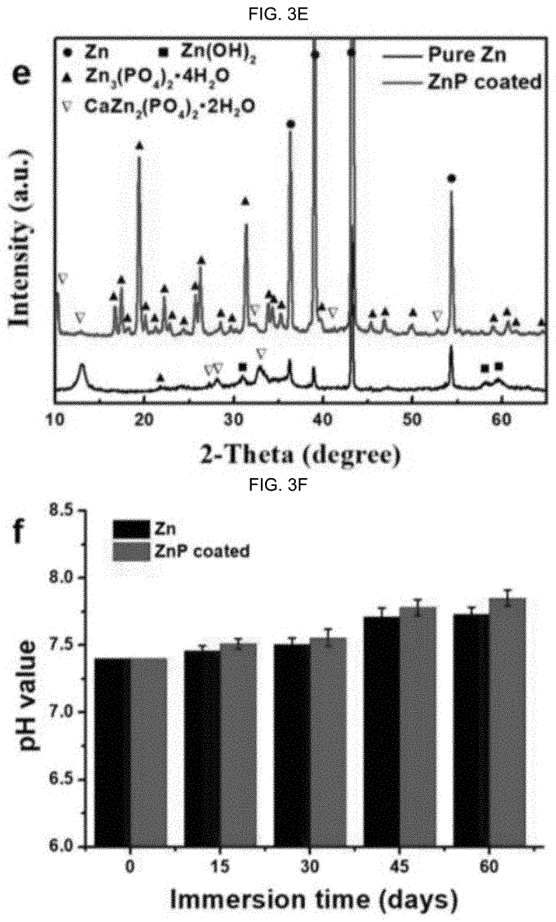

In alternative embodiments, products of manufacture or coatings as provided herein have coating stability; e.g., during the degradation in vivo they can degrade uniformly, thus gradually providing a stable short-time biocompatibility and pH stability for the degradable metal alloys, and long-term protection and biocompatibility for non-degradable metal alloys, as illustrated in FIG. 3. FIG. 3A-F illustrate degraded surface morphology and compositions of ZnP-coated surfaces and non-coated surfaces after two months of immersion test in the Hanks' solution: (a, c) general view and (b, d) magnified view of (a, b) uncoated Zn surface and (c, d) ZnP coated surface, and the corresponding (e) X-ray diffraction pattern (XRD) patterns; (f) pH evolution with immersion time.

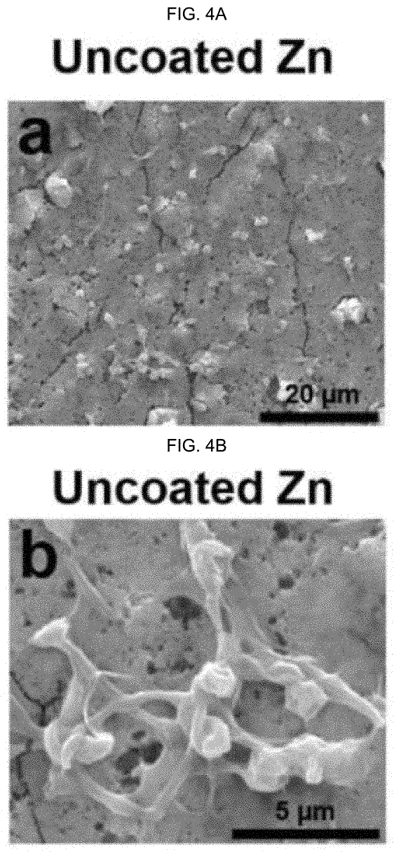

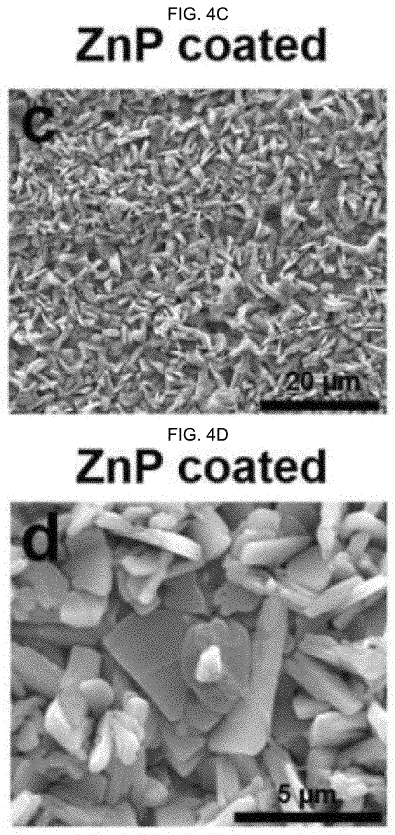

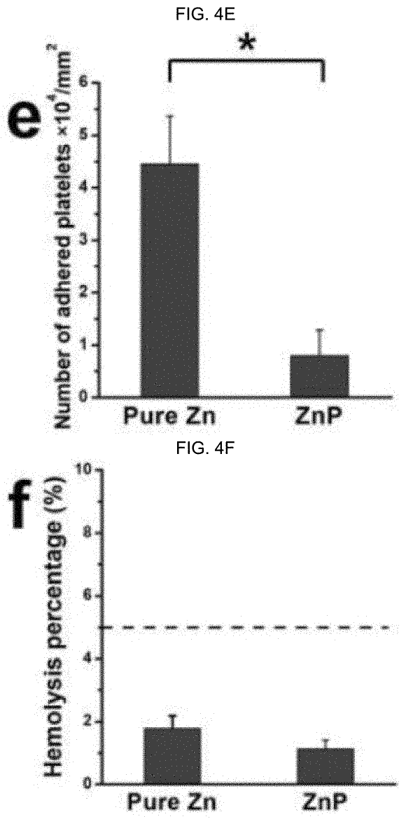

In alternative embodiments, surface ZnP-based coatings as provided herein have good hemocompatibility, as evidenced by decreased platelets adhesion at the sample surface, which significantly keeps the hemolysis rate under 5%, as illustrated in FIG. 4. FIG. 4A-F illustrate the hemocompatibility of ZnP-coated surfaces and non-coated surfaces samples: (a-d) Platelets adhesion morphology on (a, b) pure Zn, (c, d) ZnP coating, and (e) the corresponding number of adhered platelets, (f) hemolysis percentage. *p<0.05, compared between groups.

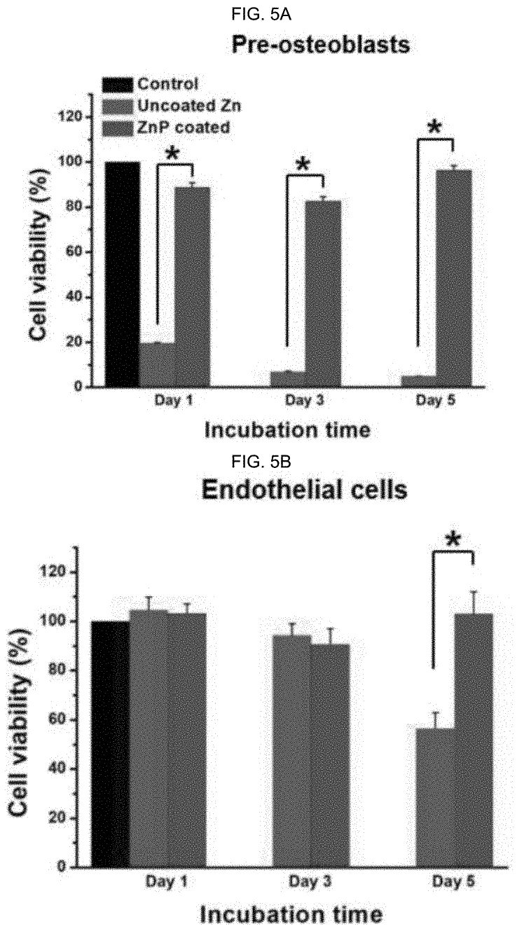

In alternative embodiments, surface ZnP based coatings as provided herein have good cytocompatibility to significantly improve the surface cell viability, as compared to non-coated substrate materials. For example, the ZnP coating can improve cell viability on a Zn surface from 10 to 20% to 80 to 100% for pre-osteoblasts, and from 55% to 105% at day 5 for endothelial cells, as illustrated in FIG. 5. FIG. 5A-B graphically illustrates data from an MTT (3-(4,5-dimethylthiazol-2-yl)-2,5-diphenyltetrazolium bromide) assay for cell viability: (FIG. 5A) pre-osteoblasts and (FIG. 5B) endothelial cells cultured with extract media prepared by incubation with samples for 72 h; *p<0.05, compared between groups.

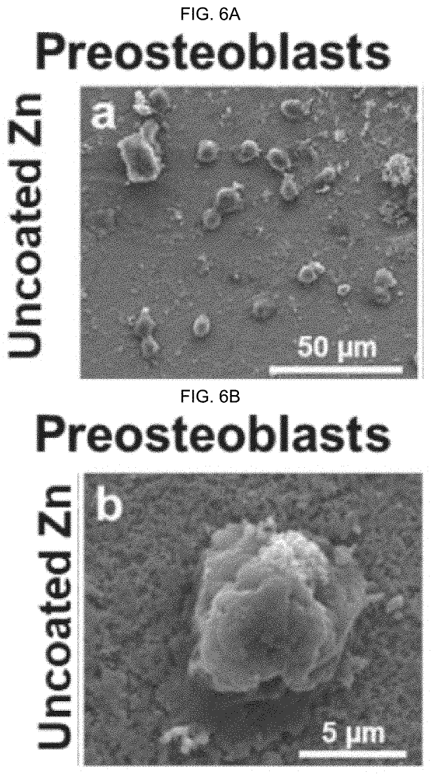

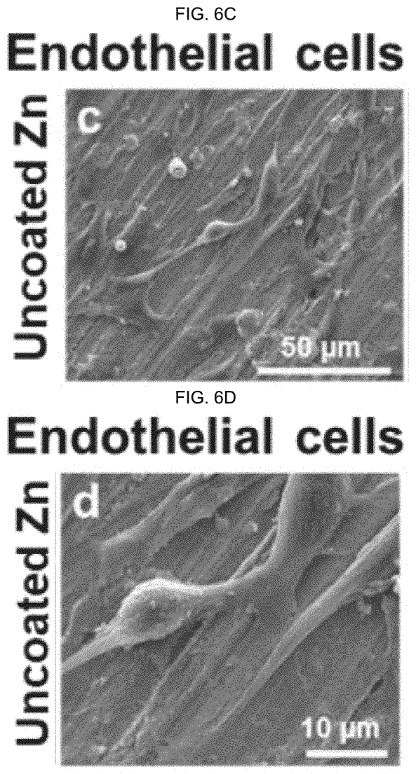

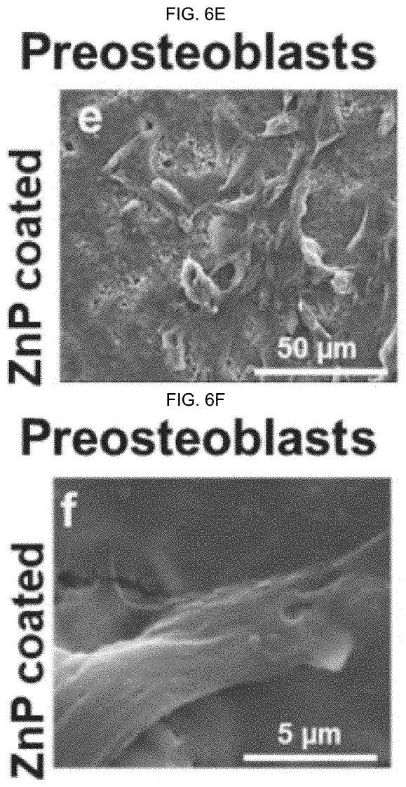

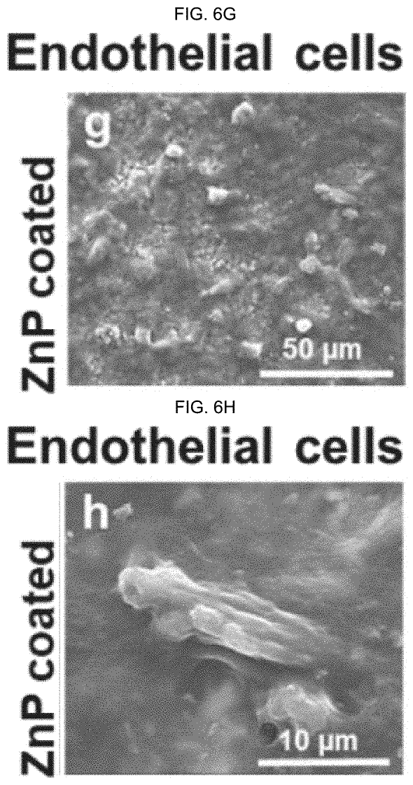

In alternative embodiments, surface ZnP based coatings as provided herein have good cytocompatibility to improve cell (e.g., pre-osteoblasts) adhesion and cell spreading on a material's surface, as compared to non-coated substrate materials, as illustrated in FIG. 6. FIG. 6A-H illustrate images showing cell adhesion morphology of (a, b, e, f) pre-osteoblasts and (c, d, g, h) endothelial cells with different samples for 72 h: (a-d) uncoated Zn, (e-h) ZnP coating.

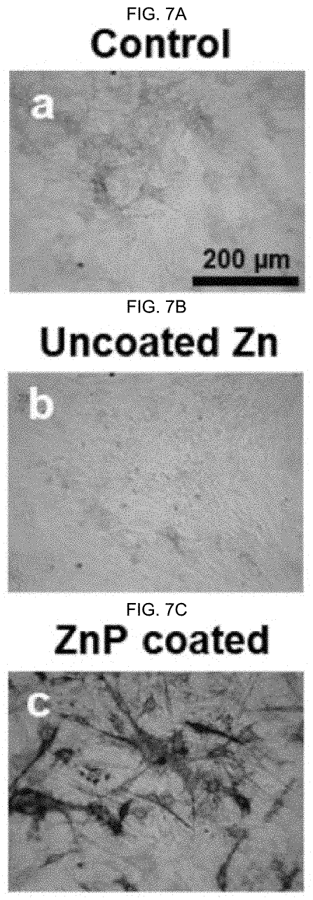

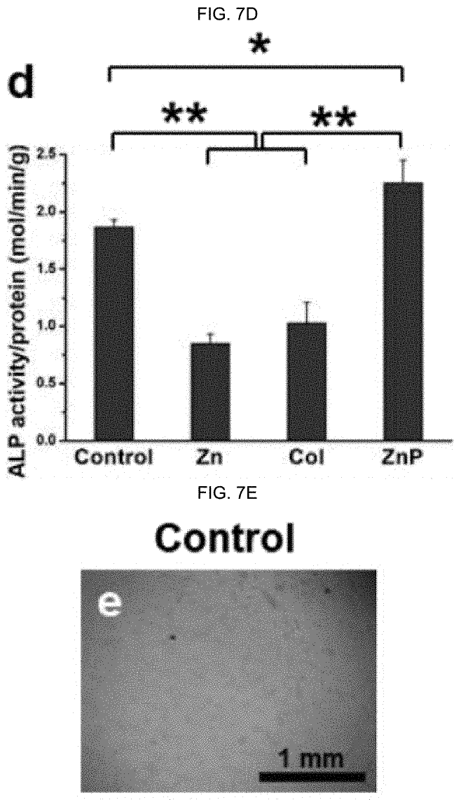

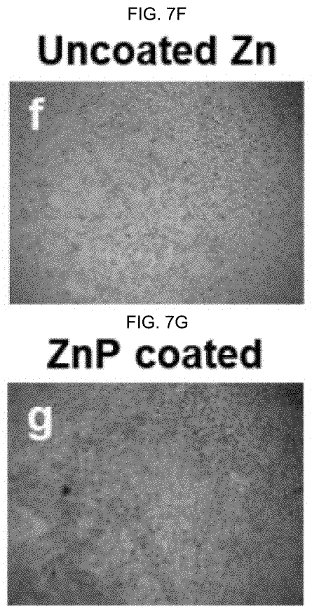

In alternative embodiments, surface ZnP based coatings as provided herein have good cytocompatibility to improve the differentiation of osteoblast cells, e.g. improve alkaline phosphatase (ALP) activity and calcific deposition of pre-osteoblasts, as illustrated in FIG. 7. FIG. 7A-G illustrates images of different cell differentiation behavior of pre-osteoblasts cultured with different sample extracts, depending on whether the substrate was coated or uncoated: (a-c) ALP staining and (d) ALP activity and (e-g) alkaline red staining: (a, e) negative control, (b, f) uncoated Zn, (c, g) ZnP coating. *p<0.05, **p<0.005, compared between groups.

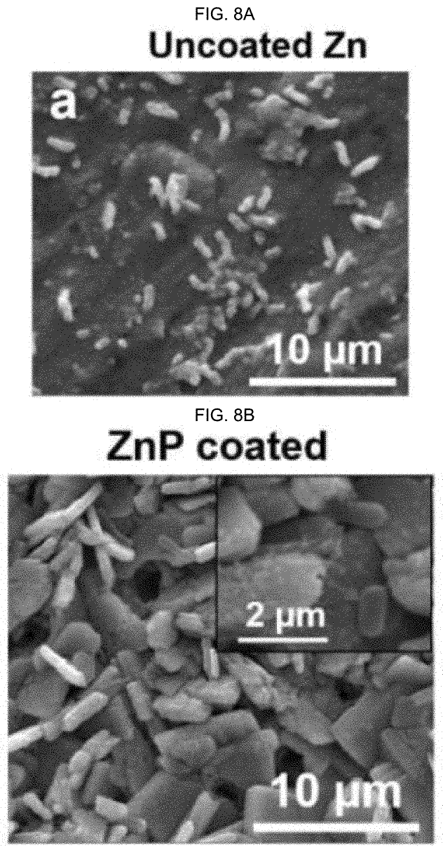

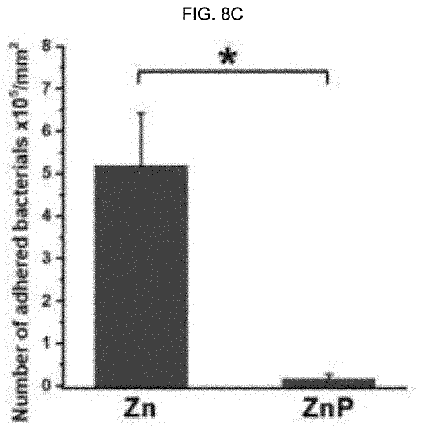

In alternative embodiments, surface ZnP based coatings as provided herein have good antibacterial properties to significantly decrease or prevent surface adhesion bacteria to the substrate, as illustrated in FIG. 8, which presents one example of the significantly decreased E. coli adhesion on the Zn surface after ZnP coating. FIG. 8A-C illustrate antibacterial performance of different samples cultured with E. coli for 24 hours (h); bacterial adhesion on surfaces of: (a) uncoated Zn, (b) ZnP coating, and (c) the corresponding number of adhered bacterial cells. *p<0.05, compared between groups.

### REFERENCES

1. Chen, Q. & Thouas, G. A. Metallic implant biomaterials. Materials Science and Engineering: R: Reports 87, 1-57 (2015).
2. Wang, X. et al. Topological design and additive manufacturing of porous metals for bone scaffolds and orthopaedic implants: a review. Biomaterials 83, 127-141 (2016).
3. Bønaa, K. H. et al. Drug-eluting or bare-metal stents for coronary artery disease. New England Journal of Medicine 375, 1242-1252 (2016).
4. Bowen, P. K. et al. Biodegradable Metals for Cardiovascular Stents: from Clinical Concerns to Recent Zn-Alloys. Adv Healthc Mater 5, 1121-1140 (2016).
5. Zheng, Y. F., et al, Biodegradable metals. Materials Science and Engineering: R: Reports 77, 1-34 (2014).
6. Heublein, B. et al. Biocorrosion of magnesium alloys: a new principle in cardiovascular implant technology? Heart 89, 651-656 (2003).
7. Campoccia, D., et al, The significance of infection related to orthopedic devices and issues of antibiotic resistance. Biomaterials 27, 2331-2339 (2006).
8. Chen, Y., et al, Recent advances on the development of magnesium alloys for biodegradable implants. Acta Biomater. 10, 4561-4573 (2014).
9. Zhao, N. & Zhu, D. Application of Mg-based alloys for cardiovascular stents. Int. J. Biomed. Eng. Technol. 12, 382-398 (2013).
10. Zhao, N. & Zhu, D. Endothelial responses of magnesium and other alloying elements in magnesium-based stent materials. Metallomics 7, 118-128 (2015).
11. Ma, J., Zhao, N. & Zhu, D. Biphasic responses of human vascular smooth muscle cells to magnesium ion. Journal of biomedical materials research. Part A 104, 347-356 (2016).
12. McCall, K. A., et al, Function and mechanism of zinc metalloenzymes. The Journal of nutrition 130, 1437S-1446S (2000).
13. Su, Y. et al. Zinc-Based Biomaterials for Regeneration and Therapy. Trends Biotechnol. (2018).
14. Zhu, D. et al. Zinc regulates vascular endothelial activities through zinc-sensing receptor ZnR/GPR39. American Journal of Physiology-Cell Physiology 314, C404-C414 (2018).
15. Bowen, P. K., et al, Zinc Exhibits Ideal Physiological Corrosion Behavior for Bioabsorbable Stents. Advanced Materials 25, 2577-2582 (2013).
16. Vojtech, D., et al, Mechanical and corrosion properties of newly developed biodegradable Zn-based alloys for bone fixation. Acta Biomater. 7, 3515-3522 (2011).
17. Trumbo, P., et al, Dietary reference intakes: vitamin A, vitamin K, arsenic, boron, chromium, copper, iodine, iron, manganese, molybdenum, nickel, silicon, vanadium, and zinc. J Am Diet Assoc 101, 294-301 (2001).
18. Zhang, D., et al, Cellular responses of osteoblast-like cells to 17 elemental metals. Journal of biomedical materials research. Part A 105, 148-158 (2017).
19. Zhu, D. et al. Biological Responses and Mechanisms of Human Bone Marrow Mesenchymal Stem Cells to Zn and Mg Biomaterials. ACS Appl Mater Interfaces 9, 27453-27461 (2017).
20. Li, H. F. et al. Development of biodegradable Zn-1× binary alloys with nutrient alloying elements Mg, Ca and Sr. Scientific reports 5, 10719 (2015).
21. Shearier, E. R. et al. In Vitro Cytotoxicity, Adhesion, and Proliferation of Human Vascular Cells Exposed to Zinc. Acs Biomater Sci Eng 2, 634-642 (2016).
22. Jarcho, M. Calcium phosphate ceramics as hard tissue prosthetics. Clinical Orthopaedics and Related Research® 157, 259-278 (1981).
23. Dorozhkin, S. V. Calcium orthophosphate coatings on magnesium and its biodegradable alloys. Acta Biomater. 10, 2919-2934 (2014).
24. Surmenev, R. A., et al, Significance of calcium phosphate coatings for the enhancement of new bone osteogenesis—A review. Acta Biomater. 10, 557-579 (2014).
25. Su, Y., Niu, L., Lu, Y., Lian, J. & Li, G. Preparation and Corrosion Behavior of Calcium Phosphate and Hydroxyapatite Conversion Coatings on AM60 Magnesium Alloy. J. Electrochem. Soc. 160, C536-0541 (2013).
26. Su, Y. et al. Enhancing the corrosion resistance and surface bioactivity of a calcium-phosphate coating on a biodegradable AZ60 magnesium alloy via a simple fluorine post-treatment method. RSC Advances 5, 56001-56010 (2015).
27. Su, Y. et al. Improvement of the Biodegradation Property and Biomineralization Ability of Magnesium-Hydroxyapatite Composites with Dicalcium Phosphate Dihydrate and Hydroxyapatite Coatings. Acs Biomater Sci Eng 2, 818-828 (2016).
28. Su, Y., et al, Composite Microstructure and Formation Mechanism of Calcium Phosphate Conversion Coating on Magnesium Alloy. J. Electrochem. Soc. 163, G138-G143 (2016).
29. Su, Y. et al. Development and characterization of silver containing calcium phosphate coatings on pure iron foam intended for bone scaffold applications. Mater Design 148, 124-134 (2018).
30. Su, Y. et al. Improving the Degradation Resistance and Surface Biomineralization Ability of Calcium Phosphate Coatings on a Biodegradable Magnesium Alloy via a Sol-Gel Spin Coating Method. J. Electrochem. Soc. 165, C155-C161 (2018).
31. Herschke, L., et al, Zinc phosphate as versatile material for potential biomedical applications Part 1. Journal of Materials Science: Materials in Medicine 17, 81-94 (2006).
32. Herschke, L., et al, Zinc phosphate as versatile material for potential biomedical applications Part II. Journal of Materials Science: Materials in Medicine 17, 95-104 (2006).
33. Zhao, X.-c., Xiao, G.-y., Zhang, X., Wang, H.-y. & Lu, Y.-p. Ultrasonic induced rapid formation and crystal refinement of chemical conversed hopeite coating on titanium. The Journal of Physical Chemistry C 118, 1910-1918 (2014).
34. Adhilakshmi, A., et al, Protecting electrochemical degradation of pure iron using zinc phosphate coating for biodegradable implant applications. New J. Chem. (2018).
35. Niu, L., J et al, A study and application of zinc phosphate coating on AZ91D magnesium alloy. Surf. Coat. Technol. 200, 3021-3026 (2006).
36. Wang, Y.-W. et al. Superior antibacterial activity of zinc oxide/graphene oxide composites originating from high zinc concentration localized around bacteria. ACS Appl. Mat. Interfaces 6, 2791-2798 (2014).

A number of embodiments of the invention have been described. Nevertheless, it can be understood that various modifications may be made without departing from the spirit and scope of the invention. Accordingly, other embodiments are within the scope of the following claims.

What is claimed is:

1. A product of manufacture comprising a zinc phosphate (ZnP) or a ZnP-based composite coating, or a combination of a ZnP coating and a ZnP-based composite coating, on or deposited on:
(a) a zinc (Zn) metal surface, or a Zn-based metal or Zn alloy surface;
(b) a magnesium (Mg) or an iron (Fe) surface, or an Mg-based or an iron (Fe)-based metal surface or Mg or Fe alloy surface; or,
(c) a nondegradable metal or nondegradable metal alloy surface, wherein optionally the nondegradable metal or nondegradable metal alloy surface comprises stainless steel, a titanium (Ti) or Ti alloy, a cobalt-chrome alloy, a nickel titanium alloy, or a combination thereof.

2. The product of manufacture of claim 1, manufactured as a medical or a dental device, wherein optionally the device is a bone implant or a prosthetic, or the device is an orthopedic, dental, craniofacial or cardiovascular device, or is used for a biomedical application, wherein optionally the biomedical application is an orthopedic, dental, craniofacial or cardiovascular application.

3. The product of manufacture of claim 1, wherein the ZnP or the ZnP-based composite coating is porous or non-porous, or a combination thereof, wherein optionally the porous ZnP surface is made or deposited by a process comprising hydrothermal, anodization, electrochemical deposition or any combination thereof; and optionally the non-porous ZnP coating is made or deposited by a process comprising spray deposition, pulsed laser deposition, sputter deposition or any combination thereof.

4. The product of manufacture of claim 1, wherein the ZnP coating or the ZnP-based composite coating is (or averages): between about 0.5 μm to 100 μm, or between about 1.0 μm to 50 μm, where the average ZnP or ZnP-based composite coating thickness on a surface using a chemical method of deposition is between about 3 μm to 6 μm.

5. The product of manufacture of claim 1, wherein the ZnP coating or the ZnP-based composite coating covers between about 1% to 100%, or between about 10% and 90%, or between about 20% and 80%, of the surface of the product of manufacture.

6. The product of manufacture of claim 1, wherein the ZnP-based composite coating comprises one or more other (non-Zn) inorganic coating compositions, wherein optionally the one or more other (non-Zn) inorganic coating compositions comprise zinc oxide, zinc hydroxide, a calcium phosphate, a hydroxyapatite (HA), or an equivalent or a mixture thereof,
wherein optionally the inorganic component of the ZnP composite coating comprises between about 1% to 95%, or between about 10% and 90%, or between about 20% and 80%, by weight, mass or molar ratio of the ZnP composite coating (the remainder being ZnP).

7. The product of manufacture of claim 1, wherein the ZnP-based composite coating comprises one or more organic compounds, herein optionally the one or more organic compounds comprise a collagen, chitosan, gelatin, hyaluronic acid, polylactic acid, polylactide, or an equivalent or a mixture thereof,
wherein optionally the organic component of the ZnP composite coating comprises between about 1% to 95%, or between about 10% and 90%, or between about 20% and 80%, by weight, mass or molar ratio of the ZnP composite coating (the remainder being ZnP).

8. The product of manufacture of claim 1, wherein the ZnP coating or the ZnP-based composite coating is a multi-layered structure, wherein optionally the inner layer of the coating comprises a Mg alloy, wherein optionally the Mg alloy comprises magnesium hydroxide and/or magnesium phosphate.

9. The product of manufacture of claim 1, further comprising a cell, wherein optionally the cell is a pre-osteoblast or an osteoblast.

10. The product of manufacture of claim 1, wherein the Zn alloy comprises aluminum, iron, magnesium, calcium, strontium, silver, copper, titanium, manganese or lithium or any combination thereof; and optionally the Zn alloy has a proportion of the following compositions:
from zero to about 12.0 weight percent of aluminum, or between about 0 to about 25 weight percent of aluminum,
from zero to about 10.0 weight percent of magnesium, or between about 0 to about 20 weight percent of magnesium,
from zero to about 10.0 weight percent of calcium, or between about 0 to about 20 weight percent of calcium,
from zero to about 10.0 weight percent of strontium, or between about 0 to about 20 weight percent of strontium,
from zero to about 8.0 weight percent of silver, or between about 0 to about 20 weight percent of silver,
from zero to about 8.0 weight percent of copper, or between about 0 to about 20 weight percent of copper,
from zero to about 5.0 weight percent of titanium, or between about 0 to about 20 weight percent of titanium,
from zero to about 5.0 weight percent of manganese, or between about 0 to about 20 weight percent of manganese,
from zero to about 5.0 weight percent of lithium, or between about 0 to about 20 weight percent of lithium,
and a balance of zinc, based on the total weight of the composition.

11. The product of manufacture of claim 1, wherein the magnesium (Mg) alloy comprises aluminum, zinc, calcium, strontium, silver, copper, titanium, manganese or lithium or any combination thereof; and optionally has a proportion of the following compositions:
from zero to about 12.0, or between about 0 to about 25, weight percent of aluminum,
from zero to about 10.0, or between about 0 to about 20, weight percent of zinc,
from zero to about 10.0, or between about 0 to about 20, weight percent of calcium,
from zero to about 10.0, or between about 0 to about 20, weight percent of strontium,
from zero to about 8.0, or between about 0 to about 20, weight percent of silver,
from zero to about 8.0, or between about 0 to about 20, weight percent of copper,
from zero to about 5.0, or between about 0 to about 20, weight percent of titanium,
from zero to about 5.0, or between about 0 to about 20, weight percent of manganese,
from zero to about 5.0, or between about 0 to about 20, weight percent of lithium,
and a balance of zinc, based on the total weight of the composition.

12. The product of manufacture of claim 1, wherein the iron (Fe) alloy comprises aluminum, zinc, calcium, strontium, silver, copper, titanium, manganese or lithium or any combination thereof; and optionally has a proportion of the following compositions:
from zero to about 40.0, or between about 0 to about 60, weight percent of manganese,
from zero to about 10.0, or between about 0 to about 20, weight percent of cobalt,
from zero to about 8.0, or between about 0 to about 20, weight percent of aluminum,
from zero to about 8.0, or between about 0 to about 20, weight percent of tungsten,
from zero to about 8.0, or between about 0 to about 20, weight percent of tin,
from zero to about 8.0, or between about 0 to about 20, weight percent of boron,
from zero to about 8.0, or between about 0 to about 20, weight percent of carbon,
from zero to about 5.0, or between about 0 to about 20, weight percent of sulfur,
from zero to about 5.0, or between about 0 to about 20, weight percent of silicon,
and a balance of zinc, based on the total weight of the composition.

13. The product of manufacture of claim 1, wherein the ZnP content of the ZnP-comprising coatings is between about 50.1% to 100%, or between about 55% and 95%, or between about 60% and 90%, by weight of the ZnP-comprising coating.

14. A kit comprising a product of manufacture of claim 1, optionally comprising instructions for using the product of manufacture of claim 1.

15. A method for increasing cell adhesion to a product of manufacture in situ or in vivo, comprising: implanting in vivo a product of manufacture of claim 1.

16. A method for enhancing vascularization in situ or in vivo, comprising: implanting in vivo a product of manufacture of claim 1.

17. A method for increasing the rate of osteogenic differentiation of pre-osteoblasts to osteoblasts, comprising: implanting in vivo a product of manufacture of claim 1.
